(19) United States
(12) Patent Application Publication
(10) Pub. No.: US 2022/0354815 A1
(43) Pub. Date: Nov. 10, 2022

(54) OKN-007 AS A THERAPEUTIC AGENT

(71) Applicant: Oklahoma Medical Research Foundation, Oklahoma City, OK (US)

(72) Inventor: Rheal A. Towner, Piedmont, OK (US)

(21) Appl. No.: 17/774,200

(22) PCT Filed: Nov. 19, 2020
(86) PCT No.: PCT/US2020/061284

§ 371 Date: May 4, 2022

Related U.S. Application Data
(60) Provisional application No. 62/937,663, filed on Nov. 19, 2019.

(51) Int. Cl. A61K 31/205; A61K 31/282; A61K 31/337; A61P 35/00

(52) U.S. Cl. CPC A61K 31/205; A61K 31/282; A61K 31/337; A61P 35/00

(57) ABSTRACT

The present invention includes compositions and methods for treating a proliferative disease in a patient that comprises administering to the patient a therapeutically effective amount of 2,4-Disulfonyl-N-Tert-Butylnitrone, 2,4-disulfonyl α-phenyl tertiary butyl nitron or a pharmaceutically acceptable salt thereof.

### TECHNICAL FIELD OF THE INVENTION

The present invention relates in general to the field of a therapeutic agent for the treatment of multiple cancers, cancer metastasis, or amyotrophic lateral sclerosis.

### STATEMENT OF FEDERALLY FUNDED RESEARCH

Not Applicable.

### BACKGROUND OF THE INVENTION

Without limiting the scope of the invention, its background is described in connection with cancer therapy and metastasis.

The present inventors have previously demonstrated that OKN-077 increases temozolomide (TMZ) sensitivity and suppresses TMZ-Resistant Glioblastoma (GBM) tumor growth. Towner, et al., Translational Oncology, Volume 12, Number 2, Feb. 2019 pp. 320-335. Understanding that treatment of glioblastoma (GBM) remains a challenge using conventional chemotherapy, such as temozolomide (TMZ), and is often ineffective as a result of drug resistance, the inventors have previously assessed a novel nitrone-based agent, OKN-700, and found it to be effective in decreasing tumor volumes and increasing survival in orthotopic GBM xenografts by decreasing cell proliferation and angiogenesis and increasing apoptosis.

However, a need remains for novel agents that can be used more broadly to treat cancers, and more particularly, the use of single agents to treat cancer, prevent metastasis, and treat cancers are secondary locations.

### SUMMARY OF THE INVENTION

In one embodiment, the present invention includes a method for treating a proliferative disease in a patient that comprises administering to the patient a therapeutically effective amount of 2,4-Disulfonyl-N-Tert-Butylnitrone, 2,4-disulfonyl α-phenyl tertiary butyl nitrone or a pharmaceutically acceptable salt thereof In one aspect, the proliferative disease is selected from at least one of an endometrial, cholangicarcinoma, lung carcinoma, non-small cell lung cancer, colon, colorectal, uterine, ovarian, pancreatic duct adenocarcinoma, or pancreatic cancer. In another aspect, the therapeutically effective amount of 2,4-Disulfonyl-N-Tert-Butylnitrone, 2,4-disulfonyl α-phenyl tertiary butyl nitrone or a pharmaceutically acceptable salt thereof inhibits an epithelial-mesenchymal transition (EMT) of cells. In another aspect, the 2,4-Disulfonyl-N-Tert-Butylnitrone, 2,4-disulfonyl α-phenyl tertiary butyl nitrone or a pharmaceutically acceptable salt thereof inhibits cancer cell metastasis. In another aspect, the 2,4-Disulfonyl-N-Tert-Butylnitrone, 2,4-disulfonyl α-phenyl tertiary butyl nitrone or a pharmaceutically acceptable salt thereof is administered orally, intravenously, or intraperitoneally. In another aspect, the 2,4-Disulfonyl-N-Tert-Butylnitrone, 2,4-disulfonyl α-phenyl tertiary butyl nitrone or a pharmaceutically acceptable salt thereof are from about 40 to 1,200 mg/kg body weight/day, 100 to 450 mg/kg body weight/day, 200 to 400 mg/kg body weight/day, 300 to 800 mg/kg body weight/day, 350 to 1,000 mg/kg body weight/day, or 400 to 1,100 mg/kg body weight/day. In another aspect, the 2,4-Disulfonyl-N-Tert-Butylnitrone, 2,4-disulfonyl α-phenyl tertiary butyl nitrone or a pharmaceutically acceptable salt thereof is at least one of: (1) administered at least one of continuously, intermittently, systemically, or locally, (2) administered one or more times a day for as long as the patient is in need of treatment for the proliferative disease; (3) sequentially or concomitantly, with another pharmaceutical agent in a newly diagnosed proliferative disease patient, to maintain remission, or a relapsed/refractory proliferative disease patient; (4) as a single agent or in combination with another pharmaceutical agent in a newly diagnosed proliferative disease patient, to maintain remission, or a relapsed/refractory proliferative disease patient; (5) as a single agent or in combination with another pharmaceutical agent in a newly diagnosed proliferative disease pediatric patient, to maintain remission, or a relapsed/refractory proliferative disease patient; or (6) a pediatric patient. In another aspect, the patient is relapsed/refractory to a chemotherapy. In another aspect, the 2,4-Disulfonyl-N-Tert-Butylnitrone, 2,4-disulfonyl α-phenyl tertiary butyl nitrone or a pharmaceutically acceptable salt thereof inhibits immune suppression against the proliferative disease. In another aspect, the proliferative disease is not a glioblastoma. In another aspect, the proliferative disease is a secondary tumor. In another aspect, the 2,4-Disulfonyl-N-Tert-Butylnitrone, 2,4-disulfonyl α-phenyl tertiary butyl nitrone, or a pharmaceutically acceptable salt thereof, is provided in a sustained-release formulation. In another aspect, the 2,4-Disulfonyl-N-Tert-Butylnitrone, 2,4-disulfonyl α-phenyl tertiary butyl nitrone, or a pharmaceutically acceptable salt thereof, is formulated or administered together, separately or sequentially with a chemotherapy or an adjuvant therapy. In another aspect, the 2,4-Disulfonyl-N-Tert-Butylnitrone, 2,4-disulfonyl α-phenyl tertiary butyl nitrone, or a pharmaceutically acceptable salt thereof, is effective against the proliferative disease that utilizes the transforming growth factor beta 1 pathway to promote tumor growth.

In another embodiment, the present invention includes a method of treating a patient with a cancer comprising: identifying that the patient is in need for treatment for the cancer; and providing an amount of 2,4-Disulfonyl-N-Tert-Butylnitrone, 2,4-disulfonyl α-phenyl tertiary butyl nitrone, or a pharmaceutically acceptable salt thereof, effective to treat the cancer, wherein the cancer is not a glioblastoma. In one aspect, the cancer is selected from at least one of an endometrial, cholangicarcinoma, lung carcinoma, non-small cell lung cancer, colon, colorectal, ovarian, uterine, pancreatic duct adenocarcinoma, or pancreatic cancer. In another aspect, the 2,4-Disulfonyl-N-Tert-Butylnitrone, 2,4-disulfonyl α-phenyl tertiary butyl nitrone or a pharmaceutically acceptable salt thereof inhibits an epithelial-mesenchymal transition (EMT) of cells. In another aspect, the 2,4-Disulfonyl-N-Tert-Butylnitrone, 2,4-disulfonyl α-phenyl tertiary butyl nitrone or a pharmaceutically acceptable salt thereof inhibits cancer cell metastasis. In another aspect, the 2,4-Disulfonyl-N-Tert-Butylnitrone, 2,4-disulfonyl α-phenyl tertiary butyl nitrone or a pharmaceutically acceptable salt thereof is administered orally, intravenously, or intraperitoneally. In another aspect, the 2,4-Disulfonyl-N-Tert-Butylnitrone, 2,4-disulfonyl α-phenyl tertiary butyl nitrone or a pharmaceutically acceptable salt thereof are from about 40 to 1,200 mg/kg body weight/day, 100 to 450 mg/kg body weight/day, 200 to 400 mg/kg body weight/day, 300 to 800 mg/kg body weight/day, 350 to 1,000 mg/kg body weight/day, or 400 to 1,100 mg/kg body weight/day. In another aspect, the 2,4-Disulfonyl-N-Tert-Butylnitrone, 2,4-disulfonyl α-phenyl tertiary butyl nitrone or a pharmaceutically acceptable salt thereof is at least one of: (1) administered at least one of continuously, intermittently, systemically, or locally, (2) administered one or more times a day for as long as the patient is in need of treatment for the cancer; (3) sequentially or concomitantly, with another pharmaceutical agent in a newly diagnosed proliferative disease patient, to maintain remission, or a relapsed/refractory cancer patient; (4) as a single agent or in combination with another pharmaceutical agent in a newly diagnosed cancer patient, to maintain remission, or a relapsed/refractory proliferative disease patient; (5) as a single agent or in combination with another pharmaceutical agent in a newly diagnosed pediatric cancer patient, to maintain remission, or a relapsed/refractory proliferative disease cancer patient; or (6) a pediatric patient. In another aspect, the patient is relapsed/refractory to a chemotherapy. In another aspect, the 2,4-Disulfonyl-N-Tert-Butylnitrone, 2,4-disulfonyl α-phenyl tertiary butyl nitrone or a pharmaceutically acceptable salt thereof inhibits immune suppression against the cancer. In another aspect, the cancer is a secondary tumor. In another aspect, the 2,4-Disulfonyl-N-Tert-Butylnitrone, 2,4-disulfonyl α-phenyl tertiary butyl nitrone, or a pharmaceutically acceptable salt thereof, is provided in a sustained-release formulation. In another aspect, the 2,4-Disulfonyl-N-Tert-Butylnitrone, 2,4-disulfonyl α-phenyl tertiary butyl nitrone, or a pharmaceutically acceptable salt thereof, is formulated or administered together, separately or sequentially with a chemotherapy or an adjuvant therapy. In another aspect, the 2,4-Disulfonyl-N-Tert-Butylnitrone, 2,4-disulfonyl α-phenyl tertiary butyl nitrone, or a pharmaceutically acceptable salt thereof, is effective against the cancer that utilizes the transforming growth factor beta 1 pathway to promote tumor growth.

In another embodiment, the present invention includes a method for treating a patient with a cancer comprising: obtaining a sample from the patient; determining if the patient has a cancer that is causing an immune suppression; and administering a therapeutically effective amount of a therapeutically effective amount of 2,4-Disulfonyl-N-Tert-Butylnitrone, 2,4-disulfonyl α-phenyl tertiary butyl nitrone or a pharmaceutically acceptable salt thereof sufficient to overcome the immune suppression caused by the cancer. In one aspect, the cancer is selected from at least one of an endometrial, cholangicarcinoma, lung carcinoma, non-small cell lung cancer, colon, colorectal, uterine, ovarian, pancreatic duct adenocarcinoma, or pancreatic cancer. In another aspect, the therapeutically effective amount of 2,4-Disulfonyl-N-Tert-Butylnitrone, 2,4-disulfonyl α-phenyl tertiary butyl nitrone or a pharmaceutically acceptable salt thereof inhibits an epithelial-mesenchymal transition (EMT) of cells. In another aspect, the 2,4-Disulfonyl-N-Tert-Butylnitrone, 2,4-disulfonyl α-phenyl tertiary butyl nitrone or a pharmaceutically acceptable salt thereof inhibits cancer cell metastasis. In another aspect, the therapeutically effective amount of 2,4-Disulfonyl-N-Tert-Butylnitrone, 2,4-disulfonyl α-phenyl tertiary butyl nitrone or a pharmaceutically acceptable salt thereof is administered orally, intravenously, or intraperitoneally. In another aspect, the 2,4-Disulfonyl-N-Tert-Butylnitrone, 2,4-disulfonyl α-phenyl tertiary butyl nitrone, or a pharmaceutically acceptable salt thereof, is provided in a sustained-release formulation. In another aspect, the 2,4-Disulfonyl-N-Tert-Butylnitrone, 2,4-disulfonyl α-phenyl tertiary butyl nitrone, or a pharmaceutically acceptable salt thereof, is formulated or administered together, separately or sequentially with a chemotherapy or an adjuvant therapy. In another aspect, the 2,4-Disulfonyl-N-Tert-Butylnitrone, 2,4-disulfonyl α-phenyl tertiary butyl nitrone, or a pharmaceutically acceptable salt thereof, is effective against the cancer that utilizes the transforming growth factor beta 1 pathway to promote tumor growth. In another aspect, the 2,4-Disulfonyl-N-Tert-Butylnitrone, 2,4-disulfonyl α-phenyl tertiary butyl nitrone or a pharmaceutically acceptable salt thereof are from about 40 to 1,200 mg/kg body weight/day, 100 to 450 mg/kg body weight/day, 200 to 400 mg/kg body weight/day, 300 to 800 mg/kg body weight/day, 350 to 1,000 mg/kg body weight/day, or 400 to 1,100 mg/kg body weight/day.

In another embodiment, the present invention includes a method of treating or reducing symptoms of amyotrophic lateral sclerosis (ALS) or a related motor neuron disorder in a subject suffering thereof, the method comprising administering to the subject an effective amount of a therapeutically effective amount of 2,4-Disulfonyl-N-Tert-Butylnitrone, 2,4-disulfonyl α-phenyl tertiary butyl nitrone or a pharmaceutically acceptable salt thereof sufficient to treat or reduce the symptoms of amyotrophic lateral sclerosis (ALS) or a related motor neuron disorder. In one aspect, the composition reduces motor neuron degeneration in the subject. In another aspect, the 2,4-Disulfonyl-N-Tert-Butylnitrone, 2,4-disulfonyl α-phenyl tertiary butyl nitrone, or a pharmaceutically acceptable salt thereof, is administered with a pharmaceutically acceptable carrier or excipient. In another aspect, the 2,4-Disulfonyl-N-Tert-Butylnitrone, 2,4-disulfonyl α-phenyl tertiary butyl nitrone, or a pharmaceutically acceptable salt thereof, is administered repeatedly to the subject. In another aspect, the 2,4-Disulfonyl-N-Tert-Butylnitrone, 2,4-disulfonyl α-phenyl tertiary butyl nitrone, or a pharmaceutically acceptable salt thereof, is provided in a sustained-release formulation. In another aspect, the 2,4-Disulfonyl-N-Tert-Butylnitrone, 2,4-disulfonyl α-phenyl tertiary butyl nitrone, or a pharmaceutically acceptable salt thereof, is provided in an amount from about 40 to 1,200 mg/kg body weight/day, 100 to 450 mg/kg body weight/day, 200 to 400 mg/kg body weight/day, 300 to 800 mg/kg body weight/day, 350 to 1,000 mg/kg body weight/day, or 400 to 1,100 mg/kg body weight/day. In another aspect, the 2,4-Disulfonyl-N-Tert-Butylnitrone, 2,4-disulfonyl α-phenyl tertiary butyl nitrone, or a pharmaceutically acceptable salt thereof, is administered orally, intravenously, or intraperitoneally. In another aspect, the related motor neuron disorder is a disorder selected from the group consisting of primary lateral sclerosis, progressive muscular atrophy, pseudobulbar palsy and progressive bulbar palsy, and fronto temporal dementia.

### BRIEF DESCRIPTION OF THE DRAWINGS

For a more complete understanding of the features and advantages of the present invention, reference is now made to the detailed description of the invention along with the accompanying figures and in which:

FIG. 1 shows the study design of the method of the present invention.

FIGS. 2A to 2E shows the results from treatment with OKN-077 slows down the disease progression, but does not delay the disease onset. FIG. 2A shows disease progression scoring system with examples of how stage 0 and 2 look like. FIG. 2B shows the treatment with OKN-077 doesn't delay the disease onset. FIG. 2C shows that the treatment with OKN-077 slows down disease progression, as the treated G93A mice reach the score of 2 around 14 days later than untreated G93A mice. FIG. 2D shows that the treated G93A mice did not progress beyond the score of 2, while untreated G93A mice reached the score of 3 around 136 days of age. FIG. 2E shows a summary of disease progression — disease scores at the time of mice sacrifice.

FIGS. 3A to 3C show that the treatment with OKN-077 didn't affect body weight (FIG. 3A), fat mass (FIG. 3B), lean mass (QMR) (FIG. 3C), nor muscle mass.

FIGS. 4A and 4B show that treatment with OKN-077 prevents motor neuron loss. FIG. 4A shows a representative image of a ventral horn of spinal cord cross-section stained for NeuN. FIG. 4B shows Motor neuron (MN) counts: there is a treatment effect (two-way ANOVA, p=0.0016), and a significant difference in MN number between treated and untreated G93A mice (Sidak's post hoc test, p=0.0197.

FIGS. 5A to 5C show that OKN-077 decreases microglia, but not astrocyte activation. FIG. 5A shows that there is genotype (p<0.0001) and treatment effect (p=0.0244) on microglia proliferation, as the number of Ibal+cells changes with gentotype and treatment. OKN-077 decreases also microglia activation, since there is significantly more ramified, non-activated microglia in treated G93A mice comparing to untreated ones (Sidak's post hoc test, p=0.0026). FIGS. 5B and 5C shows that OKN-077 doesn't affect astrocyte proliferation (two-way ANOVA, p=0.0016, source of variation: genotype p<0.0001, treatment p=0.23).

FIGS. 6A to 6C show that OKN-077 affects tissue structure (ADC), blood flow, and metabolite levels. FIG. 6A shows diffusion-weighted MRI revealed changes in apparent diffusion coefficient (ADC) values: OKN-007-treated G93A (ALS t) mice had a significantly decreased ADC in the lumbar spinal cord compared to non-treated G93A (ALS c) mice, while perfusion MRI. FIG. 6B shows that OKN-007-treated G93A mice had significantly increased relative tissue blood flow (rTBF). FIG. 6C shows that MR spectroscopy (MRS) revealed a significant decrease in myo-inositol (Myo-Ins)/creatine ratios for ALS c vs ALS t mice.

FIG. 7 is a graph that shows treatment of endometrial cancer in vivo as the survival demonstrating that there was a significant increase in survival OKN-007-treated tumor-bearing mice, compared to untreated (UT).

FIG. 8 shows MR images depicting tumors in the mid-tumor region (maximal tumor) for each treatment group (OKN-077 (OKN), or untreated (UT).

FIG. 9 is a graph that shows tumor volumes of endometrial cancer compared at the same time-point that the UT mice were terminated (at˜21 days after cell implantation). The OKN-077 treatment groups (p<0.0001) were found to significantly decrease tumor volumes compared to UT mice.

FIG. 10 is a graph that shows the results of treating endometrial cancer with OKN-077 combined with carboplatin (50 mg/kg weekly; i.p.) and paclitaxel (15 mg/kg weekly; i.p.)

FIG. 11 is a graph that shows the results of endometrial cancer tumor volumes were compared at the same time-point that the UT mice were terminated (at˜21 days after cell implantation).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

The present invention includes various methods of treating patients with compound 2,4-Disulfonyl-N-Tert-Butylnitrone, 2,4-disulfonyl α-phenyl tertiary butyl nitrone, also known as: OKN-700, NXY-059, 2,4-DSPBN, 2,4-disulfonyl phenyl tert-butyl nitrone, 2,4-ds-PBN and Cerovive.

OKN-077 is a small molecule which can easily reach several solid and metastatic tumors, and has been demonstrated to have no adverse effects on normal cells, unlike several chemotherapeutic agents that have systemic toxicity. This agent can be effective against several cancers that utilize the transforming growth factor beta 1 pathway to promote tumor growth.

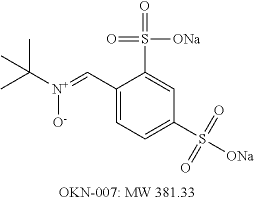

In addition to affecting cell proliferation, angiogenesis and apoptosis, OKN-077 also could potentially reduce metastasis as it downregulates CD44v6, a biomarker which is directly associated with metastasis. Therefore, OKN-077 affects all aspects of tumor growth. There are very few drugs, if any, that are effective against all four indications of tumor growth (cell proliferation, angiogenesis, apoptosis and metastasis). OKN-077 can be administered either orally in pill form (patient compliant), or intravenously.

Further, it was found that OKN-077 is an anti-inflammatory agent, and disease progression of ALS. There are currently no seems to protect motor neurons, as well as inhibit late-stage therapies that have these qualities. All other therapeutic approaches do not prevent late-stage disease progression.

Cancer. Cancer is a group of diseases involving abnormal cell growth with the potential to invade or spread to other parts of the body. These contrast with benign tumors, which do not spread to other parts of the body. Possible signs and symptoms include a lump, abnormal bleeding, prolonged cough, unexplained weight loss and a change in bowel movements. While these symptoms may indicate cancer, they may have other causes. Over 100 types of cancers affect humans. The present invention includes the treatment of cancers including, e.g., endometrial, cholangicarcinoma, lung carcinoma, non-small cell lung cancer, colon, colorectal, uterine, ovarian, pancreatic duct adenocarcinoma, or pancreatic cancer.

Cancer can spread from its original site by local spread, lymphatic spread to regional lymph nodes or by hematogenous spread via the blood to distant sites, known as metastasis. When cancer spreads by a hematogenous route, it usually spreads all over the body. However, cancer ‘seeds’ grow in certain selected site only ('soil') as hypothesized in the soil and seed hypothesis of cancer metastasis. The symptoms of metastatic cancers depend on the tumor location and can include enlarged lymph nodes (which can be felt or sometimes seen under the skin and are typically hard), enlarged liver or enlarged spleen, which can be felt in the abdomen, pain or fracture of affected bones and neurological symptoms.

The therapeutic methods of the present invention (which include prophylactic treatment) in general include administration of a therapeutically effective amount of the compositions described herein to a subject in need thereof, including a mammal, particularly a human Such treatment will be suitably administered to subjects, particularly humans, suffering from, having, susceptible to, or at risk for cancer, or having a symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

The cancer may be a carcinoma, sarcoma, lymphoma, leukemia, melanoma, mesothelioma, multiple myeloma, or seminoma. In some embodiments, the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, ovarian, pancreas, prostate, skin, spleen, small intestine, large intestine, stomach, testicle, or thyroid. In some embodiments, the cancer is leukemia, non-small cell lung cancer, colon cancer, CNS cancer, melanoma, ovarian cancer, renal cancer, breast cancer, or prostate cancer.

By way of explanation, but not a limitation of the present invention, it is believed that the mechanism-of-action of OKN-077 is inhibition of the transforming growth factor beta 1 (TGF-β1 ) pathway, which has a direct effect on the epidermal-mesenchymal transition (EMT) and metastasis, both associated with aggressive forms of malignant cancers. OKN-077 specifically affects genes that are regulated by the TGF-β1 pathway. The TGF-β1 gene is not directly down-regulated, but protein expression is decreased. Many of the genes down-regulated genes are associated with the extracellular matrix (ECM), and therefore affects cell invasion directly.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention.

As used herein, the term “therapeutically effective amount” is meant an amount of a compound of the present invention effective to yield the desired therapeutic response. For example, an amount effective to delay the growth of or to cause a cancer, to shrink or not metastasize. The specific safe and effective amount or therapeutically effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

As used herein, a “pharmaceutical salt” is salt for making an acid or base salts of a compounds. Examples of pharmaceutically acceptable salts include, but are not limited to, mineral or organic acid salts of basic residues such as amines; alkali or organic salts of acidic residues such as phenols. Preferably the salts are made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like. The preferred phenolate salts are the alkaline earth metal salts, sodium, potassium or lithium.

As used herein, a “pharmaceutical carrier” is a pharmaceutically acceptable solvent, suspending agent or vehicle, for delivering the OKN-077 compound to the animal or human. The carrier may be liquid or solid and is selected with the planned manner of administration in mind. Liposomes are also a pharmaceutical carrier.

OKN-077 is typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the OKN-077 may be formulated to provide, e.g., maximum and/or consistent dosing for the particular form for oral, rectal, topical, intravenous injection or parenteral administration. While the OKN-077 may be administered alone, it will generally be provided in a stable salt form mixed with a pharmaceutically acceptable carrier. The carrier may be solid or liquid, depending on the type and/or location of administration selected.

Techniques and compositions for making useful dosage forms using the present invention are described in one or more of the following references: Anderson, Philip O..; Knoben, James E.; Troutman, William G, eds., HANDBOOK OF CLINICAL DRUG DATA, Tenth Edition, McGraw-Hill, 2002; Pratt and Taylor, eds., PRINCIPLES OF DRUG ACTION, Third Edition, Churchill Livingston, New York, 1990; Katzung, ed., BASIC AND CLINICAL PHARMACOLOGY, Ninth Edition, McGraw Hill, 2007; Goodman and Gilman, eds., THE PHARMACOLOGICAL BASIS OF THERAPEUTICS, Tenth Edition, McGraw Hill, 2001; REMINGTON'S PHARMACEUTICAL SCIENCES, 20th Ed., Lippincott Williams & Wilkins., 2000, and updates thereto; Martindale, THE EXTRA PHARMACOPOEIA, Thirty-Second Edition (The Pharmaceutical Press, London, 1999); all of which are incorporated by reference, and the like, relevant portions incorporated herein by reference.

For example, the OKN-077 may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with an non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

OKN-077 may be administered in the form of liposome delivery systems, e.g., small unilamellar vesicles, large unilamellar vesicles, and multilamellar vesicles, whether charged or uncharged. Liposomes may include one or more: phospholipids (e.g., cholesterol), stearylamine and/or phosphatidylcholines, mixtures thereof, and the like.

OKN-077 may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the OKN-007 may be coupled one or more biodegradable polymers to achieve controlled release of the OKN-700, biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the OKN-077 and powdered carriers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration of OKN-077 in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration of the OKN-077 may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water-soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in REMINGTON'S PHARMACEUTICAL SCIENCES, Mack Publishing Company, a standard reference text in this field, relevant portions incorporated herein by reference.

Capsules. Capsules with OKN-077 may be prepared by filling standard two-piece hard gelatin capsules each with 10 to 500 milligrams of powdered active ingredient, 5 to 150 milligrams of lactose, 5 to 50 milligrams of cellulose and 6 milligrams magnesium stearate.

Soft Gelatin Capsules. A mixture of OKN-077 is dissolved in a digestible oil such as soybean oil, cottonseed oil or olive oil. The active ingredient is prepared and injected by using a positive displacement pump into gelatin to form soft gelatin capsules containing, e.g., 100-500 milligrams of the active ingredient. The capsules are washed and dried.

Tablets. A large number of tablets are prepared by conventional procedures so that the dosage unit of OKN-700, e.g., 1-500 milligrams of OKN-077, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

To provide an effervescent tablet appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution. A parenteral composition suitable for administration by injection is prepared by stirring 1.5% by weight of active ingredient in deionized water and mixed with, e.g., up to 10% by volume propylene glycol and water. The solution is made isotonic with sodium chloride and sterilized using, e.g., ultrafiltration.

Suspension. An aqueous suspension is prepared for oral administration so that each 5 ml contain 100 mg of finely divided active ingredient, 200 mg of sodium carboxymethyl cellulose, 5 mg of sodium benzoate, 1.0 g of sorbitol solution, U.S.P., and 0.025 ml of vanillin

### Example 1—Therapeutic Agent for Cancer Treatment.

Thus, in certain embodiments the present invention includes a method for treating a proliferative disease in a patient that comprises administering to the patient a therapeutically effective amount of 2,4-Disulfonyl-N-Tert-Butylnitrone, 2,4-disulfonyl α-phenyl tertiary butyl nitrone or a pharmaceutically acceptable salt thereof. Examples of proliferative diseases include at least one of endometrial, cholangicarcinoma, lung carcinoma, non-small cell lung cancer, colon, colorectal, uterine, ovarian, pancreatic duct adenocarcinoma, or pancreatic cancer. Further, the invention also includes using a therapeutically effective amount of 2,4-Disulfonyl-N-Tert-Butylnitrone, 2,4-disulfonyl α-phenyl tertiary butyl nitrone or a pharmaceutically acceptable salt thereof to inhibit an epithelial-mesenchymal transition (EMT) of cells. Thus, the invention includes using the 2,4-Disulfonyl-N-Tert-Butylnitrone, 2,4-disulfonyl α-phenyl tertiary butyl nitrone or a pharmaceutically acceptable salt thereof to inhibit cancer cell metastasis.

The active agent of the present invention, 2,4-Disulfonyl-N-Tert-Butylnitrone, 2,4-disulfonyl α-phenyl tertiary butyl nitrone or a pharmaceutically acceptable salt thereof can be adapted for various routes of administration. For example, the active agent can be administered orally, intravenously, or intraperitoneally.

The active agent of the present invention, 2,4-Disulfonyl-N-Tert-Butylnitrone, 2,4-disulfonyl α-phenyl tertiary butyl nitrone or a pharmaceutically acceptable salt thereof can be provided in various dosage forms. For example, the active agent of the present invention, 2,4-Disulfonyl-N-Tert-Butylnitrone, 2,4-disulfonyl α-phenyl tertiary butyl nitrone or a pharmaceutically acceptable salt thereof can be provided in an amount from about 40 to 1,200 mg/kg body weight/day, 100 to 450 mg/kg body weight/day, 200 to 400 mg/kg body weight/day, 300 to 800 mg/kg body weight/day, 350 to 1,000 mg/kg body weight/day, or 400 to 1,100 mg/kg body weight/day.

Further, the 2,4-Disulfonyl-N-Tert-Butylnitrone, 2,4-disulfonyl α-phenyl tertiary butyl nitrone or a pharmaceutically acceptable salt thereof can be provided to patients in a variety of regimes. For example, the active agent of the present invention, 2,4-Disulfonyl-N-Tert-Butylnitrone, 2,4-disulfonyl α-phenyl tertiary butyl nitrone or a pharmaceutically acceptable salt thereof can be: (1) administered at least one of continuously, intermittently, systemically, or locally, (2) administered up to three times or more a day for as long as the subject is in need of treatment for the proliferative disease; (3) sequentially or concomitantly, with another pharmaceutical agent in a newly diagnosed proliferative disease patient, to maintain remission, or a relapsed/refractory proliferative disease patient; (4) as a single agent or in combination with another pharmaceutical agent in a newly diagnosed proliferative disease patient, to maintain remission, or a relapsed/refractory proliferative disease patient; or (5) as a single agent or in combination with another pharmaceutical agent in a newly diagnosed proliferative disease pediatric patient, to maintain remission, or a relapsed/refractory proliferative disease patient. In one example, the patient is relapsed/refractory to a chemotherapy.

Further, the 2,4-Disulfonyl-N-Tert-Butylnitrone, 2,4-disulfonyl α-phenyl tertiary butyl nitrone or a pharmaceutically acceptable salt thereof can be used in a method in which it inhibits immune suppression against the proliferative disease. For example, a patient with cancer can be identified in which the immune response is lacking. The patient can be tested to show that their immune response is otherwise not compromised, but rather, specifically lacking in response to the specific cancer as a result of immune inhibiting or suppressing cytokines and/or lymphokines. To overcome this immune suppression, the 2,4-Disulfonyl-N-Tert-Butylnitrone, 2,4-disulfonyl α-phenyl tertiary butyl nitrone or a pharmaceutically acceptable salt thereof is provided to such a patient to allow the immune response to, once again, target the cancer or tumor. In one specific example, the proliferative disease is not a glioblastoma. In another example, the proliferative disease is a secondary tumor, which is in contrast to a primary tumor.

Thus, in certain aspects, the present invention includes a method of treating a patient with a cancer comprising: identifying that the patient is in need for treatment for the cancer; and providing an amount of 2,4-Disulfonyl-N-Tert-Butylnitrone, 2,4-disulfonyl α-phenyl tertiary butyl nitrone, or a pharmaceutically acceptable salt thereof, effective to treat the cancer, wherein the cancer is not a glioblastoma.

Thus, in certain aspects, the present invention includes a method for treating a patient with a cancer comprising: obtaining a sample from the patient; determining if the patient has a cancer that is causing an immune suppression; and administering a therapeutically effective amount of a therapeutically effective amount of 2,4-Disulfonyl-N-Tert-Butylnitrone, 2,4-disulfonyl α-phenyl tertiary butyl nitrone or a pharmaceutically acceptable salt thereof sufficient to overcome the immune suppression caused by the cancer.

Treatments Animal studies are conducted in accordance to the OMRF Institutional Animal Care and Use Committee policies, which follow NIH guidelines. For the cancer cell implantation models, cancer cells (10⁵ in 10-μl volume) are implanted. The animals are divided into two groups once tumors reached 10-20 mm³ in volume (as determined by MRI): OKN-077 treated (n =8) and untreated (UT)(n =7) groups. Rats are treated until tumors reached 200-250 mm³ in volume or for a total of 4-6 weeks. OKN-077 is administered in the drinking water (18 mg/kg; 0.018% w/v). For mouse cancer cell implantation models, 2-month-old male nude mice (Hsd:Athymic Nude-Foxnlnu mice; Harlan Inc., Indianapolis, IN) are implanted with human xenograft cancer cells (1Å˜10⁶) per ml suspended in 4μl in cell culture media. Once tumors reached 10-15 mm³ (determined via MRI), mice are treated either with OKN-077 in the drinking water (150 mg/kg; 0.20% w/v for a 20 g mouse) daily via gavage every 3 days. Mice were treated until the tumors reached, e.g., 100-150 mm³ or for a total of 4-6 weeks. For both rodent studies, OKN-077 was dissolved in water and made fresh every 2 days. Water bottles were weighed, and the amount of OKN-077 consumed per rodent was determined. No significant deviation was observed in the volume of liquid uptake of OKN-077 in these rodents. The average intake of OKN-007 was approximately 10 mg/kg/day/rat or 140-150 mg/kg/day/mouse. All groups are stratified to ensure that tumor sizes were similar before initiation of treatment.

### Example 2- Therapeutic Agent for ALS.

ALS is a debilitating disease that according to the ALS Association affects two per 100,000 people in the USA. There is no cure and the average survival time after diagnosis is three years. In this study, the inventors investigated the impact of treatment with OKN-700, an antioxidant, on ALS progression. Female Sodl G93A mutant mice, an established mouse model of ALS, and wildtype (wt) mice were treated with OKN-077 for three months starting at the age of 60 days, before the mice became symptomatic, and compared to age matched untreated controls (both transgenic and wt). Disease progression measured by monitoring disease score on a scale of 1-5 (based on hind limb paralysis and dysfunction) was delayed in the treated mice. All treated mice were at stage 2 at the time of sacrifice at 145 days (end stage disease is ˜160 days), while untreated transgenic controls had progressed to stage ≥2. There was also a treatment effect on motor neuron number. The untreated transgenic control mice had on average 10.7 ±2.1 alpha motor neurons per ventral horn, while the treated transgenic mice had 17.5 ±4.5 alpha motor neurons (a 64% increase). Treatment with OKN-077 also significantly affected tissue structure, where the apparent diffusion coefficient (ADC) was decreased in OKN-007-treated mice compared to untreated (p<0.001) in the lumbar region of the spinal cord, and increased ADC in the surrounding muscle tissue (p<0.05). Vascular alterations were also measured, indicating that the relative tissue blood flow in the spinal cord was significantly increase (p<0.01) in the treated mice vs. untreated. The spinal cord metabolite, myo-inositol, was found to be significantly elevated in the treated mice vs. untreated. These findings indicate that OKN-077 may potentially be considered as a possible therapeutic agent for ALS, and certainly warrants further investigation.

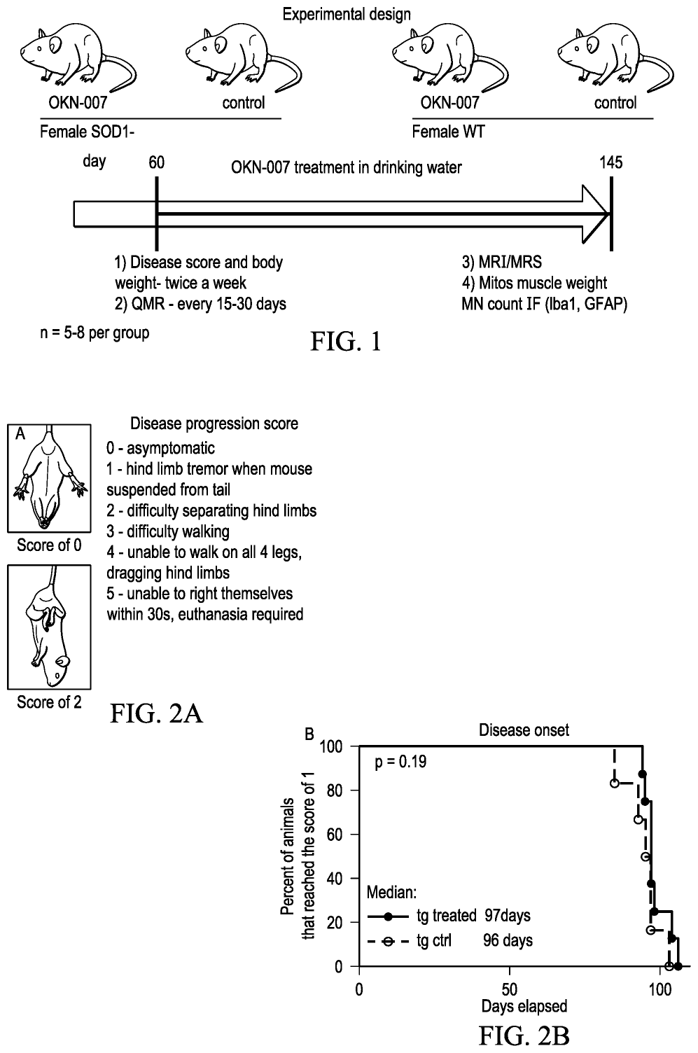

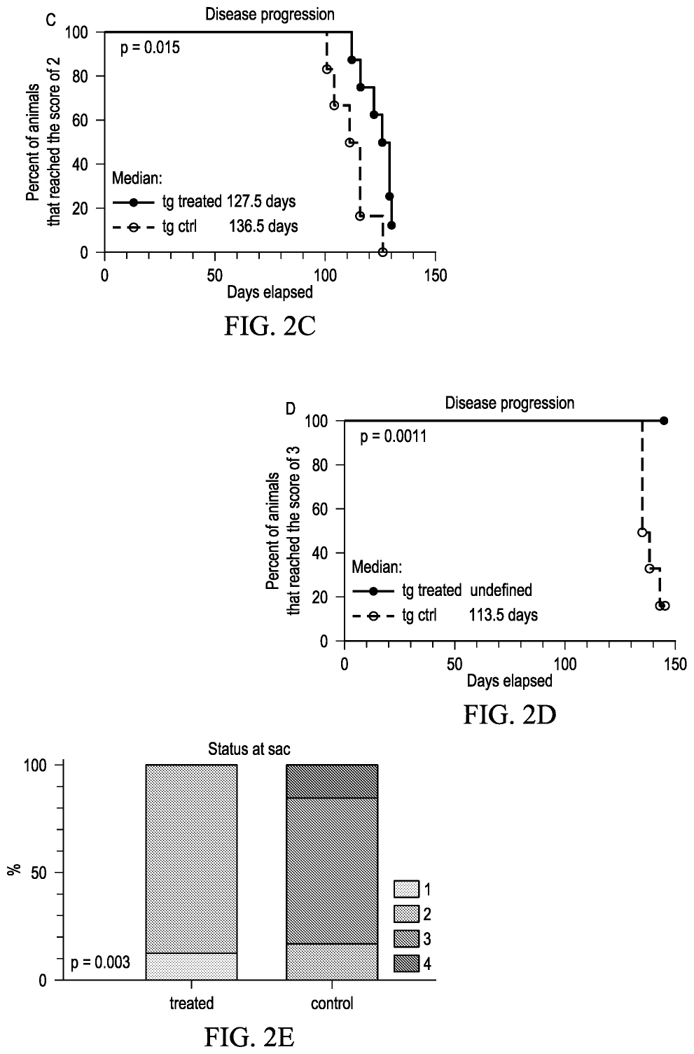

FIG. 1 shows the study design of the method of the present invention. FIGS. 2A to 2E shows the results from treatment with OKN-077 slows down the disease progression, but does not delay the disease onset. FIG. 2A shows disease progression scoring system with examples of how stage 0 and 2 look like. FIG. 2B shows the treatment with OKN-077 doesn't delay the disease onset. FIG. 2C shows that the treatment with OKN-077 slows down disease progression, as the treated G93A mice reach the score of 2 around 14 days later than untreated G93A mice. FIG. 2D shows that the treated G93A mice did not progress beyond the score of 2, while untreated G93A mice reached the score of 3 around 136 days of age. FIG. 2E shows a summary of disease progression—disease scores at the time of mice sacrifice.

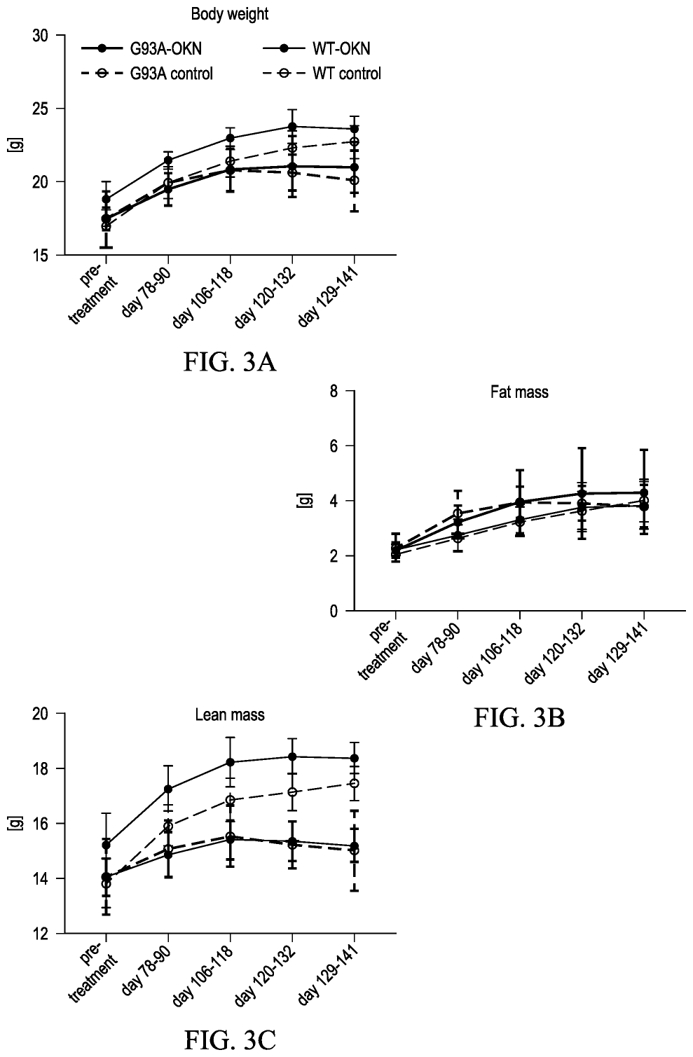

FIGS. 3A to 3C show that the treatment with OKN-077 didn't affect body weight (FIG. 3A), fat mass (FIG. 3B), lean mass (QMR) (FIG. 3C), nor muscle mass.

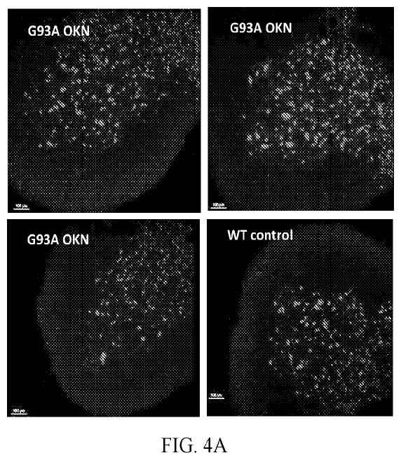

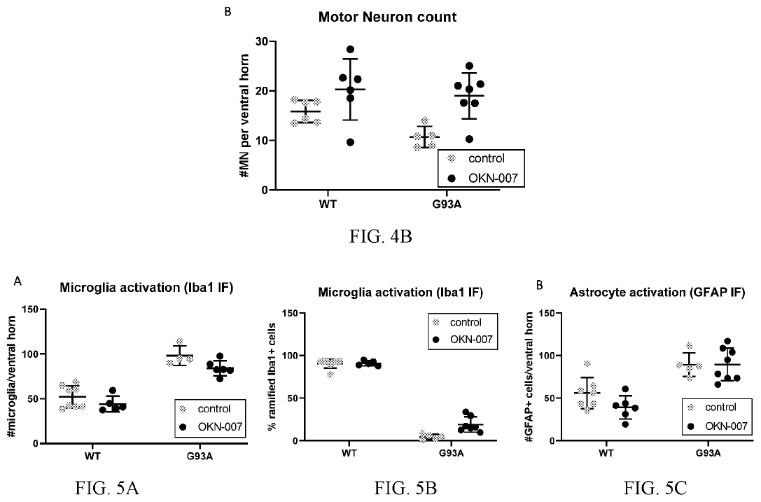

FIGS. 4A and 4B show that treatment with OKN-077 prevents motor neuron loss. FIG. 4A shows a representative image of a ventral horn of spinal cord cross-section stained for NeuN. FIG. 4B shows Motor neuron (MN) counts: there is a treatment effect (two-way ANOVA, p=0.0016), and a significant difference in MN number between treated and untreated G93A mice (Sidak's post hoc test, p=0.0197.

FIGS. 5A to 5C show that OKN-077 decreases microglia, but not astrocyte activation. FIG. 5A shows that there is genotype (p<0.0001) and treatment effect (p=0.0244) on microglia proliferation, as the number of Ibal+cells changes with gentotype and treatment. OKN-077 decreases also microglia activation, since there is significantly more ramified, non-activated microglia in treated G93A mice comparing to untreated ones (Sidak's post hoc test, p=0.0026). FIGS. 5B and 5C shows that OKN-077 doesn't affect astrocyte proliferation (two-way ANOVA, p=0.0016, source of variation: genotype p<0.0001, treatment p=0.23).

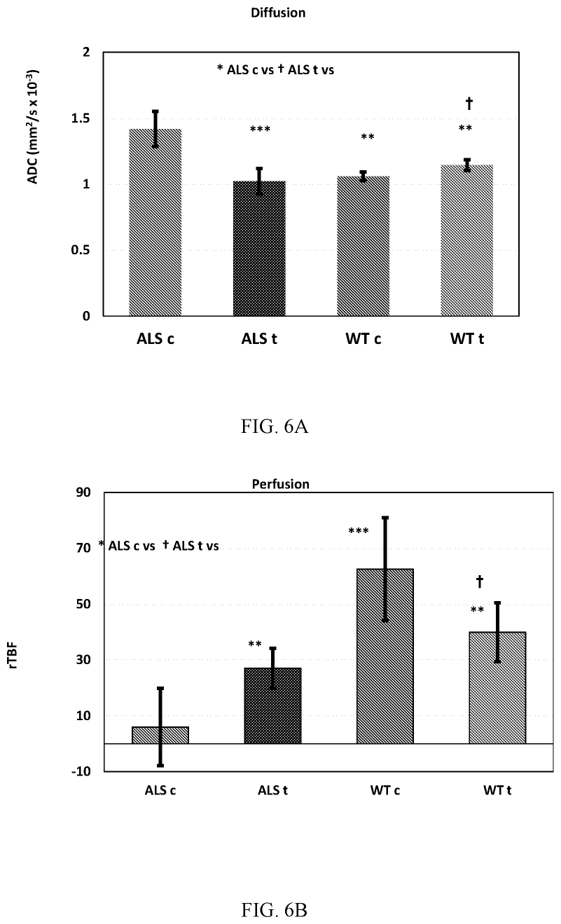

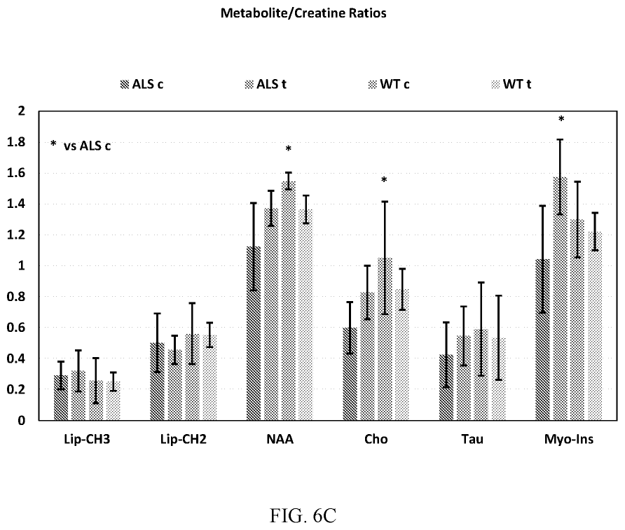

FIGS. 6A to 6C show that OKN-077 affects tissue structure (ADC), blood flow, and metabolite levels. FIG. 6A shows diffusion-weighted MRI revealed changes in apparent diffusion coefficient (ADC) values: OKN-007-treated G93A (ALS t) mice had a significantly decreased ADC in the lumbar spinal cord compared to non-treated G93A (ALS c) mice, while perfusion MRI. FIG. 6B shows that OKN-007-treated G93A mice had significantly increased relative tissue blood flow (rTBF). FIG. 6C shows that MR spectroscopy (MRS) revealed a significant decrease in myo-inositol (Myo-Ins)/creatine ratios for ALS c vs ALS t mice.

Thus, disease progression is slower in treated G93A mice. Treatment didn't affect body weight. OKN-077 decreased microglia activation, but didn't affect astrocyte activation. OKN-077 prevented motor neuron loss. OKN-077 decreased apparent diffusion coefficient (ADC) in the lumbar region of the spinal cord. The relative tissue blood flow in the spinal cord was significantly increased (P<0.01) in the treated mice vs untreated. The spinal cord metabolite, myo-inositol, was found to be significantly elevated in the treated mice vs untreated. These findings show that OKN-077 is a therapeutic agent for ALS.

### Example 3 — Treatment of Endometrial Cancer.

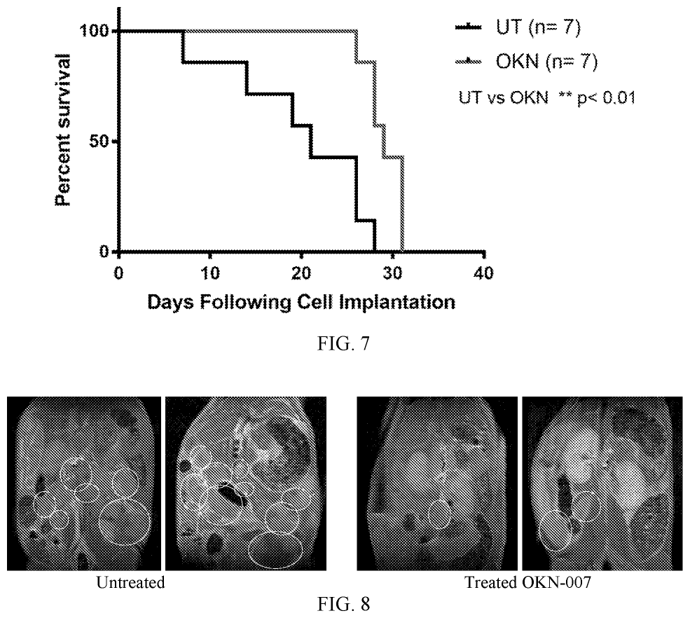

FIG. 7 is a graph that shows treatment of endometrial cancer in vivo as the survival demonstrating that there was a significant increase in survival OKN-007-treated tumor-bearing mice, compared to untreated (UT). CS99 cells were obtained from K. Moxley (OUHSC). 10⁶ cells were injected i.p. into Athymic Nude Foxnlnu mice (Envigo) mice. Gd-DTPA (MRI contrast agent; administered i.v. via a tail-vein-catheter) was used to detect tumors. OKN-077 was administered 150 mg/kg daily in drinking water. Survival indicated that there was a significant increase in OKN-007-treated tumor-bearing mice, compared to untreated (UT).

FIG. 8 shows MR images depicting tumors in the mid-tumor region (maximal tumor) for each treatment group (OKN-077 (OKN), or untreated (UT). Tumor volumes with OKN-077 had consistently small tumors at 21 days after CS99 cell implantations (compared to untreated tumors).

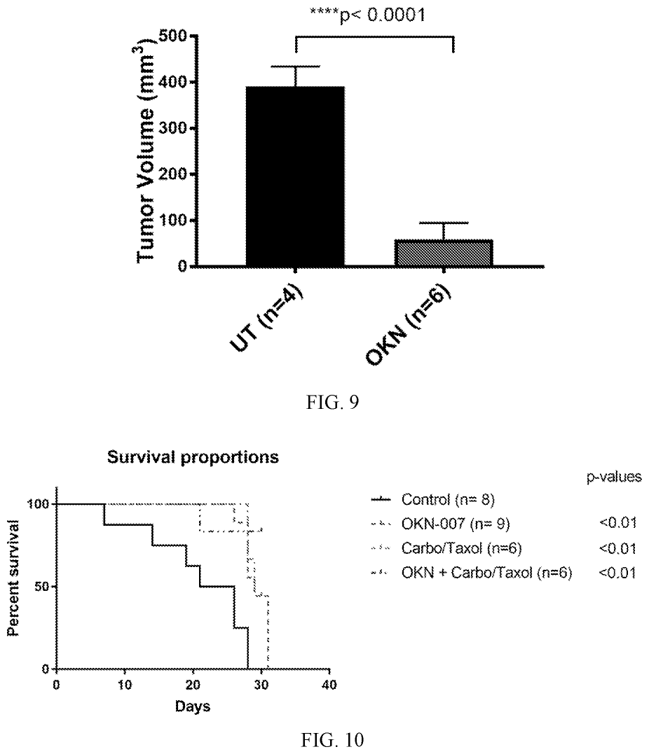

FIG. 9 is a graph that shows tumor volumes of endometrial cancer compared at the same time-point that the UT mice were terminated (at ˜21 days after cell implantation). The OKN-077 treatment groups (p<0.0001) were found to significantly decrease tumor volumes compared to UT mice.

FIG. 10 is a graph that shows the results of treating endometrial cancer with OKN-077 combined with carboplatin (50 mg/kg weekly; i.p.) and paclitaxel (15 mg/kg weekly; i.p.). All treatment groups, compared to untreated, were found to significantly increase % survival (p<0.01 for all). OKN-077 combined with carboplatin/paclitaxel resulted in 80% animal survival.

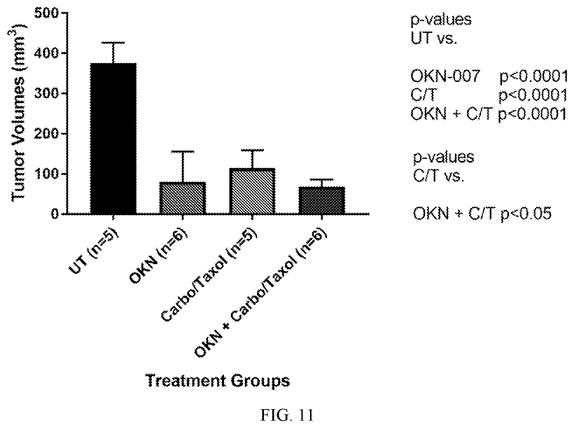

FIG. 11 is a graph that shows the results of endometrial cancer tumor volumes were compared at the same time-point that the UT mice were terminated (at ˜21 days after cell implantation). All treatment groups (p<0.0001 for all) were found to significantly decrease tumor volumes compared to UT mice. OKN-077 combined with carbo/taxol was found to be significantly lower than carbo/taxol alone (p<0.05).

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim

For each of the claims, each dependent claim can depend both from the independent claim and from each of the prior dependent claims for each and every claim so long as the prior claim provides a proper antecedent basis for a claim term or element.

1. A method for treating a proliferative disease in a patient that comprises administering to the patient a therapeutically effective amount of 2,4-Disulfonyl-N-Tert-Butylnitrone, 2,4-disulfonyl α-phenyl tertiary butyl nitrone or a pharmaceutically acceptable salt thereof.

2. The method of claim 1, wherein the proliferative disease is selected from at least one of an endometrial, cholangicarcinoma, lung carcinoma, non-small cell lung cancer, colon, colorectal, uterine, ovarian, pancreatic duct adenocarcinoma, pancreatic cancer, or is a secondary tumor.

3. The method of claim 1, wherein the therapeutically effective amount of 2,4-Disulfonyl-N-Tert- Butylnitrone, 2,4-disulfonyl α-phenyl tertiary butyl nitrone ora pharmaceutically acceptable salt thereof inhibits at least one of: an epithelial-mesenchymal transition (EMT) of cells, cancer cell metastasis, or immune suppression against the proliferative disease.

4. (canceled).

5. The method of claim 1, wherein the 2,4-Disulfonyl-N-Tert-Butylnitrone, 2,4-disulfonyl α-phenyl tertiary butyl nitrone or a pharmaceutically acceptable salt thereof is administered orally, intravenously, or intraperitoneally.

6. The method of claim 1, wherein the 2,4-Disulfonyl-N-Tert-Butylnitrone, 2,4-disulfonyl α-phenyl tertiary butyl nitrone or a pharmaceutically acceptable salt thereof are from about 40 to 1,200 mg/kg body weight/day, 100 to 450 mg/kg body weight/day, 200 to 400 mg/kg body weight/day, 300 to 800 mg/kg body weight/day, 350 to 1,000 mg/kg body weight/day, or 400 to 1,100 mg/kg body weight/day.

7. The method of claim 1, wherein the 2,4-Disulfonyl-N-Tert-Butylnitrone, 2,4-disulfonyl α-phenyl tertiary butyl nitrone or a pharmaceutically acceptable salt thereof is at least one of: (1) administered at least one of continuously, intermittently, systemically, or locally, (2) administered one or more times a day for as long as the patient is in need of treatment for the proliferative disease; (3) administered sequentially or concomitantly, with another pharmaceutical agent in a newly diagnosed proliferative disease patient, to maintain remission, or a relapsed/refractory proliferative disease patient; (4) administered as a single agent or in combination with another pharmaceutical agent in a newly diagnosed proliferative disease patient, to maintain remission, or a relapsed/refractory proliferative disease patient; (5) administered as a single agent or in combination with another pharmaceutical agent in a newly diagnosed proliferative disease pediatric patient, to maintain remission, or a relapsed/refractory proliferative disease patient; (6) administered to a pediatric patient, or (7) administered to a patient, wherein the patient is relapsed/refractory to a chemotherapy.

8. (canceled).

9. (canceled).

10. The method of claim 1, wherein the proliferative disease is not a glioblastoma.

11. (canceled).

12. The method of claim 1, wherein the 2,4-Disulfonyl-N-Tert-Butylnitrone, 2,4-disulfonyl α-phenyl tertiary butyl nitrone, or a pharmaceutically acceptable salt thereof, is at least one of: provided in a sustained-release formulation, formulated or administered together, separately or sequentially with a chemotherapy or an adjuvant therapy, or formulated with carboplatin, paclitaxel, or both, or is effective against the proliferative disease that utilize a transforming growth factor beta 1 pathway to promote tumor growth.

13. (canceled).

14. (canceled).

15. (canceled).

16. A method of treating a patient with a cancer comprising:
identifying that the patient is in need for treatment for the cancer; and
providing an amount of 2,4-Disulfonyl-N-Tert-Butylnitrone, 2,4-disulfonyl α-phenyl tertiary butyl nitrone, or a pharmaceutically acceptable salt thereof, effective to treat the cancer, wherein the cancer is not a glioblastoma.

17. The method of claim 16, wherein the cancer is selected from at least one of an endometrial, cholangicarcinoma, lung carcinoma, non-small cell lung cancer, colon, colorectal, uterine, ovarian, pancreatic duct adenocarcinoma, pancreatic cancer, or is a secondary tumor.

18. The method of claim 16, wherein the 2,4-Disulfonyl-N-Tert-Butylnitrone, 2,4-disulfonyl α-phenyl tertiary butyl nitrone or a pharmaceutically acceptable salt thereof inhibits at least one of an epithelial-mesenchymal transition (EMT) of cells, cancer cell metastasis, or immune suppression against the proliferative disease.

19. (canceled).

20. The method of claim 16, wherein the 2,4-Disulfonyl-N-Tert-Butylnitrone, 2,4-disulfonyl α-phenyl tertiary butyl nitrone or a pharmaceutically acceptable salt thereof is administered orally, intravenously, or intraperitoneally.

21. The method of claim 16, wherein the 2,4-Disulfonyl-N-Tert-Butylnitrone, 2,4-disulfonyl α-phenyl tertiary butyl nitrone or a pharmaceutically acceptable salt thereof are from about 40 to 1,200 mg/kg body weight/day, 100 to 450 mg/kg body weight/day, 200 to 400 mg/kg body weight/day, 300 to 800 mg/kg body weight/day, 350 to 1,000 mg/kg body weight/day, or 400 to 1,100 mg/kg body weight/day.

22. The method of claim 16, wherein the 2,4-Disulfonyl-N-Tert-Butylnitrone, 2,4-disulfonyl α-phenyl tertiary butyl nitrone or a pharmaceutically acceptable salt thereof is at least one of: (1) administered at least one of continuously, intermittently, systemically, or locally, (2) administered one or more times a day for as long as the patient is in need of treatment for the cancer; (3) administered sequentially or concomitantly, with another pharmaceutical agent in a newly diagnosed proliferative disease patient, to maintain remission, or a relapsed/refractory cancer patient; (4) administered as a single agent or in combination with another pharmaceutical agent in a newly diagnosed cancer patient, to maintain remission, or a relapsed/refractory proliferative disease patient; (5) administered as a single agent or in combination with another pharmaceutical agent in a newly diagnosed pediatric cancer patient, to maintain remission, or a relapsed/refractory proliferative disease cancer patient; [ [or]] (6) administered to a pediatric patient, or (7) administered to a patient, wherein the patient is relapsed/refractory to a chemotherapy.

23. (canceled).

24. (canceled).

25. (canceled).

26. The method of claim 16, wherein the 2,4-Disulfonyl-N-Tert-Butylnitrone, 2,4-disulfonyl α-phenyl tertiary butyl nitrone, or a pharmaceutically acceptable salt thereof, is at least one of: provided in a sustained-release formulation,. formulated or administered together, separately or sequentially with a chemotherapy or an adjuvant therapy, or formulated with carboplatin, paclitaxel, or both, or is effective against the proliferative disease that utilize a transforming growth factor beta 1 pathway to promote tumor growth.

27. (canceled).

28. (canceled).

29. (canceled).

30. A method for treating a patient with a cancer comprising:
obtaining a sample from the patient;
determining if the patient has a cancer that is causing an immune suppression; and
administering a therapeutically effective amount of a therapeutically effective amount of 2,4- Disulfonyl-N-Tert-Butylnitrone, 2,4-di sulfonyl α-phenyl tertiary butyl nitrone or a pharmaceutically acceptable salt thereof sufficient to overcome the immune suppression caused by the cancer.

31. The method of claim 30, wherein the cancer is selected from at least one of an endometrial, cholangicarcinoma, lung carcinoma, non-small cell lung cancer, colon, colorectal, uterine, ovarian, pancreatic duct adenocarcinoma, or pancreatic cancer.

32. The method of claim 30, wherein the therapeutically effective amount of 2,4-Disulfonyl-N-Tert-Butylnitrone, 2,4-disulfonyl α-phenyl tertiary butyl nitrone or a pharmaceutically acceptable salt thereof inhibits at least one of: an epithelial-mesenchymal transition (EMT) of cells, cancer cell metastasis, or immune suppression against the proliferative disease.

33. (canceled).

34. The method of claim 30, wherein the therapeutically effective amount of 2,4-Disulfonyl-N-Tert-Butylnitrone, 2,4-disulfonyl α-phenyl tertiary butyl nitrone or a pharmaceutically acceptable salt thereof is administered orally, intravenously, or intraperitoneally.

35. The method of claim 30, wherein the 2,4-Disulfonyl-N-Tert-Butylnitrone, 2,4-disulfonyl α-phenyl tertiary butyl nitrone, or a pharmaceutically acceptable salt thereof, is at least one of: provided in a sustained-release formulation,. formulated or administered together, separately or sequentially with a chemotherapy or an adjuvant therapy, or formulated with carboplatin, paclitaxel, or both, or is effective against the proliferative disease that utilize a transforming growth factor beta 1 pathway to promote tumor growth.

36. The method of claim 30, wherein the 2,4-Disulfonyl-N-Tert-Butylnitrone, 2,4-disulfonyl α-phenyl tertiary butyl nitrone, or a pharmaceutically acceptable salt thereof, is provided at 40 to 1,200 mg/kg body weight/day, 100 to 450 mg/kg body weight/day, 200 to 400 mg/kg body weight/day, 300 to 800 mg/kg body weight/day, 350 to 1,000 mg/kg body weight/day, or 400 to 1,100 mg/kg body weight/day.

37. (canceled).

38. (canceled).

39. (canceled).

40. A method of treating or reducing symptoms of amyotrophic lateral sclerosis (ALS) or a related motor neuron disorder in a subject suffering thereof, the method comprising administering to the subject an effective amount of a therapeutically effective amount of 2,4-Disulfonyl-N-Tert-Butylnitrone, 2,4- disulfonyl α-phenyl tertiary butyl nitrone or a pharmaceutically acceptable salt thereof sufficient to treat or reduce the symptoms of amyotrophic lateral sclerosis (ALS) or a related motor neuron disorder.

41. The method of claim 37, wherein the 2,4-Disulfonyl-N-Tert-Butylnitrone, 2,4-disulfonyl α-phenyl tertiary butyl nitrone, or a pharmaceutically acceptable salt thereof, reduces motor neuron degeneration in the subject.

42. (canceled).

43. The method of claim 37, wherein the 2,4-Disulfonyl-N-Tert-Butylnitrone, 2,4-disulfonyl α-phenyl tertiary butyl nitrone, or a pharmaceutically acceptable salt thereof, is at least one of: administered repeatedly to the subject or in a sustained-release formulation, administered orally, intravenously, or intraperitoneally, or administered in a pharmaceutically acceptable carrier or excipient.

44. (canceled).

45. The method of claim 37, wherein the 2,4-Disulfonyl-N-Tert-Butylnitrone, 2,4-disulfonyl α-phenyl tertiary butyl nitrone, or a pharmaceutically acceptable salt thereof, is provided in an amount from about 40 to 1,200 mg/kg body weight/day, 100 to 450 mg/kg body weight/day, 200 to 400 mg/kg body weight/day, 300 to 800 mg/kg body weight/day, 350 to 1,000 mg/kg body weight/day, or 400 to 1,100 mg/kg body weight/day.

46. (canceled).

47. The method of claim 37, wherein a related motor neuron disorder is a disorder selected from the group consisting of primary lateral sclerosis, progressive muscular atrophy, pseudobulbar palsy and progressive bulbar palsy, and fronto temporal dementia.
